(19) United States
(12) Patent Application Publication
(10) Pub. No.: US 2003/0114406 A1
(43) Pub. Date: Jun. 19, 2003

(54) Hyaluronic acid microspheres for sustained gene transfer

(75) Inventor: Weiliam Chen, Coram, NY (US)

Correspondence Address: DARBY & DARBY P.C., Post Office Box 5257, New York, NY, 10150-5257, US

(73) Assignee: Collaborative Laboratories, Inc.

(21) Appl. No.: 10/277,184

(22) Filed: Oct. 21, 2002

Related U.S. Application Data
(63) Continuation of application No. 09/596,665, filed on Jun. 19, 2000, now abandoned.
(60) Provisional application No. 60/140,260, filed on Jun. 18, 1999.

(51) Int. Cl. A61K 48/00; A61K 31/715; A61K 9/16; A61K 9/50; C07H 21/04; C08B 37/00

(52) U.S. Cl. 514/44; 514/54; 424/493; 536/23.1; 536/53

(57) ABSTRACT

A microsphere composition comprising materials which include substances which provide increased safety and bioavailability of nucleic acids when used in gene therapy applications. The microspheres of the present invention include hyaluronic acid which has been derivatized with a dihydrazide, preferably adipic dihydrazide, which is crosslinked to a nucleic acid. These microspheres are useful in gene therapy applications for the treatment of a variety of medical conditions, such as myocardial ischemia. In the treatment of myocardial ischemia, the microspheres of the invention include the VEGF gene. When cardiac cells are transfected with VEGF, angiogenesis in cardiac tissue is a result. Angiogenesis in cardiac tissue is likely to provide a therapeutic effect in the treatment of myocardial ischemia in that blocked or damaged blood vessels may be bypassed by newly grown blood vessels.

The present application is a continuation of application Ser. No. 09/596,665, filed Jun. 19, 2000, which claims the benefit of U.S. Provisional Application No. 60/140,260, filed Jun. 18, 1999. Each of these prior applications is hereby incorporated herein by reference in its entirety.

### FIELD OF THE INVENTION

The present invention relates to a composition of dihydrazide derivatized hyaluronic acid/nucleic acid microspheres and their therapeutic use in the treatment of diseases, such as myocardial ischemia, by the induction of angiogenesis. The microspheres of the present invention offer several therapeutic advantages over previously developed gene transfer systems such as a low inflammatory response, biodegradability, and the need for only a single application of the micro spheres.

### BACKGROUND OF THE INVENTION

Myocardial ischemia induced by coronary obstruction can be treated by either pharmacotherapy or mechanical intervention. Pharmacotherapy relieves angina but does not alleviate coronary obstruction and has the additional disadvantage of offering only short term relief. Mechanical intervention with the aim of therapeutic myocardial revascularization includes: (i) bypass surgery, and (ii) coronary angioplasty followed by stent implantation. The former is a highly invasive surgery and the latter often results in restenosis within six months after intervention. An ideal revascularization therapy involving a non- or minimally invasive intervention that can achieve permanent revascularization has yet to be devised. Recent advances in the research of Vascular Endothelial Growth Factor (VEGF) promise new nonsurgical alternatives in the treatment of coronary obstruction induced myocardial ischemia through the auto-regeneration and/or revascularization of ischemic myocardium. VEGF is a potent mitogen for endothelial cells derived from arteries, veins and lymphatics, but lacks appreciable mitogenic activity for other cell types. The availability of VEGF to promote the generation of new collateral vessels would be of potential major therapeutic value for disorders characterized by inadequate tissue perfusion and could become an alternative to surgical reconstruction procedures.

Gene therapy promises the intracellular introduction of therapeutic genes into diseased tissues, thereby rendering cells within a region exposed to the gene transfer system capable of producing and/or excreting therapeutic protein. Such production of therapeutic protein in situ circumvents the disadvantages, including frequent administration and formulation obstacles (e.g., protein denaturation), that are often associated with using exogenously administered recombinant protein. In a recent gene therapy clinical trial in human subjects, intramuscular administration of the VEGF gene (plasmid DNA encoding VEGF₁₆₅) resulted in angiogenesis in patients with severe limb ischemia. The implication of these experimental results is that VEGF gene therapy, in a minimally invasive clinical intervention, has great potential for promoting angiogenesis in myocardial tissue and alleviating myocardial ischemia.

The success of gene therapy depends upon the development of safe and efficacious vehicles for the delivery of therapeutic genes. Viral (adenovirus and retrovirus) vectors are known to be very efficient in transfection of multiple cell types. The used of viral vectors in gene therapy, however, has been limited by their immunogenic activity (adenoviral vector) and mutagenic potential (retroviral vector). Cationic liposomes as a gene therapy vehicle have, generally, been unsuccessful in clinical use. The use of plasmid DNA vectors in gene therapy, therefore, offers several advantages over these other types of vectors in that plasmid DNA vectors are generally non-immunogenic and have low mutagenic potential.

Currently available polymeric delivery vehicles for plasmid DNA also have inherent disadvantages. Plasmid DNA has been incorporated into a nonbiodegradable polymer (polyethylene vinyl acetate, EVAc) matrix and achieved a sustained plasmid DNA release over a prolonged period of time. However, matrix DNA delivery systems are not suitable for myocardial injection because of their bulky consistency. This bulkiness makes it difficult for matrix DNA compositions to pass through the bore of a needle. Plasmid DNA has also been encapsulated in a bioerodable synthetic co-polymer of fumaric acid and sebacic acid (poly FA:SA) microspheres and successfully achieved gene expression in both cell culture and in rats. Plasmid DNA has also been incorporated into gelatin and chitosan nanospheres. These sustained plasmid DNA delivery systems have been used in gene transfer to cells in culture and also resulted in gene expression. In a potential disadvantage, no measures were taken to protect the DNA incorporated into these nano- and micro-sphere systems from potential degradation by nucleases, indicating that their in vivo performance could potentially be compromised by DNA degradation, both before and after cellular uptake. The polymers used to formulate these DNA delivery vehicles are also known to induce both inflammatory and immune response reactions in vivo, further limiting their use.

The present invention embodies, in part, derivatized hyaluronic acid microspheres that overcome many of the problems associated with other types of nucleic acid delivery systems that have been described previously.

### SUMMARY OF THE INVENTION

The present invention includes a microsphere comprising a dihydrazide derivatized hyaluronic acid crosslinked to a nucleic acid. The dihydrazide may be adipic dihydrazide. The nucleic acid may include a nucleotide sequence which is at least 70% identical to the nucleotide sequence set forth in SEQ ID NO.1 or it may encode a protein which includes an amino acid sequence which is at least 70% identical to the amino acid sequence set forth in SEQ ID NO.2. The nucleic acid of the HA-matrix system may be plasmid DNA, linear, single or double stranded DNA or RNA. The nucleic acids of the HA-matrix system may also encode VEGF or other genes whose expression leads to angiogenesis in a subject's body.

In preferred embodiments, the invention includes a microsphere comprising hyaluronic acid crosslinked with adipic dihydrazide wherein the adipic dihydrazide is further crosslinked to a nucleic acid wherein said nucleic acid has a nucleotide sequence which encodes a protein of at least 70% identity to the reference amino acid sequence set forth in SEQ ID NO.2, wherein identity is determined using the BLASTP algorithm, where the parameters are selected to give the largest match between the sequences tested, over the entire length of the reference sequence.

Furthermore, the invention includes methods of transfecting cells comprising contacting the cells with the microspheres of the invention; the cells transfected by these methods are also a part of the invention. Other methods of the invention include treating subjects, who may have a myocardial ischemia, who are in need of increased angiogenesis, including contacting the body of the subject with a microsphere of the invention.

In preferred embodiments, the invention includes a method of treating myocardial ischemia in a subject comprising contacting the heart of the subject with a microsphere of the invention comprising hyaluronic acid crosslinked with adipic dihydrazide wherein the adipic dihydrazide is further crosslinked to a plasmid whose nucleotide sequence comprises that set forth in SEQ ID NO. 1.

### BRIEF DESCRIPTION OF THE DRAWINGS

FIG. 1. Flow-chart for synthesis of hyaluronic acid DNA delivery microspheres.

FIG. 2. Light microscopic image of a microsphere preparation.

FIG. 3. Sustained release profile of DNA from two preparations of DNA-HA microspheres.

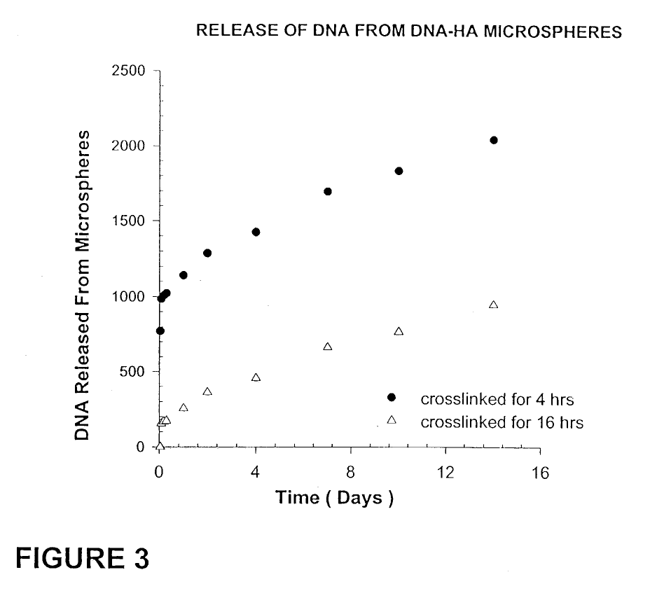

FIG. 4. Electrophoretic mobility studies of DNA-HA microspheres and DNA recovered from sustained release study.

FIG. 5. Representative area on a culture dish of CHO cells transfected with DNA samples recovered during the course of a controlled release study of DNA-HA microspheres (cross-linked for 24 hours)-β-galactosidase reporter gene used.

FIG. 6. Representative area on a culture dish of Chinese hamster ovary (CHO) cells transfected with DNA-HA microspheres (cross-linked for 4 hours)-β-galactosidase reporter gene used.

Table 1. Longevity of DNA release from DNA-HA microspheres and magnitude of CHO cell transfection.

### DETAILED DESCRIPTION

Applicant's have developed noninflammatory biodegradable and biocompatible hyaluronic acid derived microspheres that have been crosslinked with a dihydrazide for sustained transfer of plasmid DNA encoding the VEGF gene to achieve the goal of prolonged angiogenesis. The preferred dihydrazide is adipic dihydrazide. VEGF is a growth factor which strongly stimulates the growth of vascular epithelial cells. The growth of vascular epithelia is an important event in the process of angiogenesis. In contrast to other nonviral and nonliposomal experimental gene delivery vehicles, which seek to physically disperse plasmid DNA into the DNA delivery vehicles, either with or without the aid of a DNA condensing agent, and which control DNA release by limiting DNA solubility or by forming physical barriers to DNA diffusion (i.e., the delivery vehicles), the DNA of the present invention is conjugated to hyaluronic acid which has been derivatized with a dihydrazide. This mode of conjugation also renders some protection of the plasmid DNA from nucleases. Without being bound by a single theory, it is believed that the derivatized hyaluronic acid of the microspheres of the invention is degraded gradually thereby releasing nucleic acids and that on this basis the microspheres of the invention provide a sustained transfer of nucleic acids from the HA-microsphere to the cells of a subject. Without committing to a single theory, this degradation may occur by hydrolysis or by the activity of hyaluronidase enzymes. The microsphere DNA (encoding the VEGF gene) sustained delivery system will provide substantially improved prospects for coronary disease treatment through a single application; current experimental clinical protocols require multiple injections of plasmid DNA. The microspheres of this invention also provide an advantage over previously developed gene delivery systems in that the DNA is conjugated to a substance which occurs naturally in the body, hyaluronic acid. Many previously developed systems use synthetic polymers which may cause an inflammatory response.

Hyaluronic acid (HA) preparations have variable molecular weights that differ according to the purification procedure, the extent of degradation, and the source. The molecular weights may range from about 70,000 to about 4 million daltons, in a highly polymerized preparation. The hyaluronic acids are a class of macromolecular glycosaminoglycan characterized by a highly polymerized chain of glucuronic acid and N-acetylglucosamine units. HA molecules exist in nature as hydrated gels, usually closely associated with other tissue components such as chondroitin sulfate. Hyaluronic acids occur in intercellular ground tissue where they have a variety of tissue-specific vital physiological functions, including controlling tissue permeation, bacterial invasiveness, and macromolecular transport between cells. Other tissue specific functions include tissue hydration, tissue lubrication in synovial and heart valve tissue, and mechanoelectrical transduction in the vitreous humor of the eye and fluids of the inner ear. The HA carbohydrate polymer is highly negatively charged. When HA is mixed with a cationic protein such as albumin at low pH, a precipitate may be formed. Breakage of the glycosidic linkage causes depolymerization of the carbohydrate polymer, and as a consequence, no precipitation occurs. This phenomenon is the basis for the turbidimetric assay of hyaluronidase.

The term “therapeutic agent” refers to a substance, which, when delivered to a subject, causes a physiological effect in the subject.

The term “microsphere” refers to microscopic particles which include substances, such as nucleic acids, which are delivered to target cells. The substance included in a microsphere may be a therapeutic agent. In a specific embodiment, the microspheres of this invention may have a diameter of between about 15 μm and about 25 μm however, microspheres of any size wherein the essential elements of the invention are preserved are within the scope of the invention. Without being bound by theory, it is believed that the microspheres of the invention are solid, essentially homogeneous spherical bodies including dihydrazide derivatized hyaluronic acid which is crosslinked to a nucleic acid.

The term “protein” refers to any peptide or polypeptide containing two or more amino acids, modified amino acids, or amino acid derivatives. “Protein”, by way of example, and without excluding other types of proteins, includes enzymes and structural proteins.

A “DNA molecule”, “nucleic acid molecule” or “nucleic acid” refers to the phosphodiester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Double stranded DNA-DNA, DNA-RNA and RNA-RNA helices are possible. A more specific term, “oligonucleotide”, refers to a nucleic acid of 20 bases in length, or less. Thus, these terms include double-stranded DNA found, inter alia, in linear (e.g., restriction fragments) or circular DNA molecules, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA). A “recombinant DNA molecule” is a DNA molecule that has undergone a molecular biological manipulation.

A “DNA sequence” or “nucleotide sequence” is a series of nucleotide bases (also called “nucleotides”) in DNA and RNA, and means any chain of two or more nucleotides. A nucleotide sequence typically carries genetic information, including the information used by cellular machinery to make proteins. These terms include double or single stranded genomic DNA or cDNA, RNA, any synthetic and genetically manipulated nucleic acid, and both sense and anti-sense nucleic acids. This includes single- and double-stranded molecules, i.e., DNA-DNA, DNA-RNA and RNA-RNA hybrids, as well as “protein nucleic acids” (PNA) formed by conjugating bases to an amino acid backbone. This also includes nucleic acids containing modified bases, for example thio-uracil, thio-guanine and fluoro-uracil.

The term “heterologous” refers to a combination of elements not naturally occurring. For example, heterologous DNA refers to DNA not naturally located in the cell, or in a chromosomal site of the cell. Heterologous nucleic acids in a cell may include nucleic acids which include nucleotide sequences which naturally occur in the cell as well as nucleic acids which include nucleotide sequences which do not naturally occur in the cell.\ A heterologous expression regulatory element is such an element operatively associated with a different gene than the one with which it is operatively associated in nature.

The “nucleic acids” and “nucleic acid molecules” herein may be flanked by natural regulatory (expression control) sequences, or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like. The nucleic acids may also be modified by many means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.). Nucleic acids may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. The nucleic acids maybe derivatized by formation of a methyl or ethyl phosphotriester or an alkyl phosphoramidate linkage. Furthermore, the nucleic acids herein may also be modified with a label capable of providing a detectable signal, either directly or indirectly. Exemplary labels include radioisotopes, fluorescent molecules, biotin, and the like.

The term “host cell” means any cell of any organism that is selected, modified, transformed, grown, or used or manipulated in any way, for the production of a substance by the cell, for example the expression by the cell of a gene or DNA sequence.

Proteins are made in the host cell using instructions in DNA and RNA, according to the genetic code. Generally, a DNA sequence having instructions for a particular protein or enzyme is “transcribed” into a corresponding sequence of RNA. The RNA sequence in turn is “translated” into the sequence of amino acids which form the protein. Each amino acid is represented in DNA or RNA by one or more triplets of nucleotides, called a codon. The genetic code has some redundancy, also called degeneracy, meaning that most amino acids have more than one corresponding codon corresponding to an amino acid. The amino acid lysine (Lys), for example, can be coded by the nucleotide triplet or codon AAA or by the codon AAG. Codons may also form translation stop signals, of which there are three. Because the nucleotides in DNA and RNA sequences are read in groups of three for protein production, it is important to begin reading the sequence at the correct nucleotide, so that the correct triplets are read. The way that a nucleotide sequence is grouped into codons is called the “reading frame.”

The term “gene” refers to a DNA sequence that encodes or corresponds to a particular sequence of amino acids that comprise all or part of one or more proteins, and may or may not include regulatory DNA sequences, such as, for example, promoter sequences, which determine, for example, the conditions under which the gene is expressed. The term “gene” also includes DNA sequences which are transcribed from DNA to RNA, but are not translated into an amino acid sequence.

A “coding sequence” or a sequence “encoding” an expression product, such as a RNA, polypeptide, or protein, is a nucleotide sequence that, when expressed, results in the production of that RNA, polypeptide, or protein, i.e., the nucleotide sequence encodes an amino acid sequence for that polypeptide or protein. A coding sequence for a protein may include a start codon (usually ATG) and a stop codon. A nucleic acid may also “encode” a gene or DNA sequence in that the nucleotide sequence of the gene or DNA sequence is contained within the nucleic acid.

A “promoter sequence” is a DNA regulatory region capable of binding RNA polymerase in a cell and initiating transcription of a downstream (3′ direction) coding sequence. A promoter sequence is bounded typically at its 3′ terminus by the transcription initiation site and extends upstream (5′ direction) to include bases or elements necessary to initiate transcription at higher or lower levels than that of a promoter without said bases or elements. Within the promoter sequence will be found a transcription initiation site (conveniently defined, for example, by mapping with nuclease S1), as well as protein binding domains (consensus sequences) responsible for the binding of RNA polymerase.

A coding sequence is “under the control of” or “operatively associated with” transcriptional and translational control sequences in a cell when RNA polymerase transcribes the coding sequence into mRNA, which may then be spliced (if it contains introns) and may also be translated into the protein encoded by the coding sequence.

The terms “express” and “expression” mean allowing or causing the information in a gene or DNA sequence to become manifest, for example producing a protein by activating the cellular functions involved in transcription and translation of a corresponding gene or DNA sequence. A DNA sequence is expressed in or by a cell to form an “expression product” such as a protein. The expression product itself, e.g. the resulting protein, may also be said to be “expressed” by the cell. An expression product can be characterized as intracellular, extracellular or secreted. The term “intracellular” means something that is inside a cell. The term “extracellular” means something that is outside a cell. A substance is “secreted” by a cell if it appears in significant measure outside the cell, from somewhere on or inside the cell.

The term “gene transfer” refers broadly to any process by which nucleic acids are introduced into a cell. Accordingly, the term “gene therapy” refers to the use of a gene transfer process for the purpose of treating a medical condition in a subject. For the purposes of the present application, a subject or a patient may be an animal. Preferably, the subject or patient is a human.

The term “transfection” or “transformation” means the introduction of a foreign nucleic acid into a host cell. Transfection or transformation may cause the host cell to express a gene or sequence which has been introduced to produce a desired substance, typically a protein coded by the introduced gene or sequence. The introduced gene or sequence may also be called a “cloned” or “foreign” gene or sequence and may include regulatory or control sequences, such as start, stop, promoter, signal, secretion, or other sequences used by a cell's genetic machinery. The gene or sequence may include nonfunctional sequences or sequences with no known function. The DNA or RNA introduced to a host cell can come from any source, including cells of the same genus or species as the host cell, or cells of a different genus or species.

The term “vector” means the vehicle by which a DNA or RNA sequence (e.g., a foreign gene) can be introduced into a host cell, so as to transform or transfect the host. Transformation or transfection may promote expression (e.g., transcription and translation) of the introduced sequence. Vectors may include plasmids.

Vectors typically comprise the DNA of a transmissible agent, into which foreign DNA is inserted. A common way to insert one segment of DNA into another segment of DNA involves the use of enzymes called restriction enzymes, which cleave DNA at specific sites (specific groups of nucleotides) called restriction sites, and DNA ligase which joins pieces of DNA, such as a restriction enzyme digested nucleic acid and a restriction enzyme digested plasmid vector, together. A “cassette” refers to a DNA coding sequence or segment of DNA that codes for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct.” A common type of vector is a “plasmid”, which generally is a self-contained circular molecule of double-stranded DNA that can readily accept additional (foreign) DNA and which can be readily introduced into a suitable host cell. A plasmid vector often contains coding DNA and promoter DNA and has one or more restriction sites suitable for inserting foreign DNA. Promoter DNA and coding DNA may be from the same gene or from different genes, and may be from the same or different organisms. A large number of vectors, including plasmid and fungal vectors, have been described for replication and/or expression in a variety of eukaryotic and prokaryotic hosts. Non-limiting examples include pKK plasmids (Clonetech), pUC plasmids, pET plasmids (Novagen, Inc., Madison, Wis.), pRSET or pREP plasmids (Invitrogen, San Diego, Calif.), or pMAL plasmids (New England Biolabs, Beverly, Mass.), and many appropriate host cells, using methods disclosed or cited herein or otherwise known to those skilled in the relevant art. Recombinant cloning vectors will often include one or more replication systems for cloning or expression, one or more markers for selection in the host, e.g. antibiotic resistance, and one or more expression cassettes.

Accordingly, the term “sequence similarity” in all its grammatical forms refers to the degree of identity or homology between nucleic acid or amino acid sequences.

The term “sequence identity” or “identity” refers to exact matches between the nucleotides or amino acids of a two nucleic acids or proteins, respectively, when these sequences are compared. For example, the degree of sequence identity between two nucleic acids may be determined by comparison of the amino acids of these proteins by use of the BLASTN or CLUSTALW sequence comparison algorithm. Similarly, the amino acid sequences of two proteins may be determined by use of the BLASTP or CLUSTALW sequence comparison algorithm. The BLAST algorithms are publically accessible, at no cost, at the National Center for Biotechnology Information website (http://www.ncbi.nlm.nih.gov/). The CLUSTALW algorithm is publically accessible, at no cost, at the European Bioinformatics Institute website (http://www2.ebi.ac.uk/clustalw/). The present invention includes microspheres which comprise nucleic acids which have a nucleotide sequence of at least 70% identity to the reference nucleotide sequence set forth in SEQ ID NO. 1 as well as nucleic acids which have a nucleotide sequence which encodes a protein whose amino acid sequence has at least 70% identity to the reference amino acid sequence set forth in SEQ ID NO.2, wherein identity is determined using the BLASTN or BLASTP algorithms, respectively, where the parameters are selected to give the largest match between the respective sequences tested, over the entire length of the respective reference sequences. However, in preferred embodiments, the level of identity mentioned above is greater than 70%, preferably 80% or greater, more preferably 90% or greater, even more preferably 95% or greater and most preferably 100%.

As used herein, the term “sequence homology” refers to both the number of exact matches and conserved matches between the amino acid sequences of two proteins. A conserved match is a match between two amino acids which are of similar biochemical classification. For example, in the context of a protein sequence comparison, a match of one amino acid with a hydrophobic side group with a different amino acid with a hydophobic side group would be considered a conserved match. The classes which are generally known by those skilled in the art are as follows: hydrophobic (valine, leucine, isoleucine, methionine, phenylalanine, tryptophan, alanine, proline); hydrophilic (histidine, lysine, arginine, glutamic acid, aspartic acid, cysteine, asparagine, glutamine, threonine, tyrosine, serine, glycine); no charge/hydrophilic (cysteine, asparagine, glutamine, threonine, tyrosine, serine, glycine); aromatic (tryptophan, tyrosine, phenylalanine); negatively charged/hydrophilic (aspartic acid, glutamic acid); positively charged/hydrophilic (histidine, lysine, arginine).

Angiogenesis refers to the growth of new blood vessels anywhere in the body. The term “cardiac angiogenesis” refers to angiogenesis in the heart.

The term “myocardial ischemia” refers to a condition in which blood flow to cardiac tissue is reduced to a level such that the function of that tissue is impaired or may become impaired if the reduction of blood flow persists.

The term “induce” or “induction” refers to an increase by a measurable amount.

The term “derivative” refers to a compound obtained from a parent substance which includes the essential elements of said parent substance.

Dihydrazide refers to molecules having the formula: H₂N—NH—C(═O)—R—C(═O)—NH—NH₂; wherein R is a hydrocarbyl such as alkyl, aryl, alkylaryl or arylalkyl or R is heterohydrocarbyl which also includes oxygen, sulfur and/or nitrogen atoms in addition to carbon atoms. An alkyl may be branched or unbranched and contain one to 20 carbons or other carbon-sized atoms, preferably 2 to 10, more preferably 4 to 8 carbons or carbon-sized heteroatoms, such as oxygen, sulfur or nitrogen. The alkyl may be fully saturated or may contain one or more multiple bonds. The carbon atoms of the alkyl may be continuous or separated by one or more functional groups such as an oxygen atom, a keto group, an amino group, an oxycarbonyl group and the like. The alkyl may be substituted with one or more aryl groups. The alkyl may in whole or in part, be in form of rings such as cyclopentyl, cyclohexyl, and the like. These non-cyclic or cyclic groups described above may be hydrocarbyl or may include heteroatoms such as oxygen, sulfur, or nitrogen and may be further substituted with inorganic, alkyl or aryl groups including halo, hydroxy, amino, carbonyl, etc. Any of the alkyl groups described above may have double or triple bond(s). Moreover, any of the carbon atoms of the alkyl group may be separated from each other or from the dihydrazide moiety with one or more groups such as carbonyl, oxycarbonyl, amino, and also oxygen and sulfur atoms singly or in a configuration such as —S—S—,—O—CH₂,—CH.₂—O—, S—S—CH₂—CH₂—and NH(CH₂)ₙ NH—. Aryl substituents are typically substituted or unsubstituted phenyl, but may also be any other aryl group such as pyrolyl, furanyl, thiophenyl, pyridyl, thiazoyl, etc. The aryl group may be further substituted by an inorganic, alkyl or other aryl group including halo, hydroxy, amino, thioether, oxyether, nitro, carbonyl, etc. The alkylaryl or arylalkyl groups may be a combination of alkyl and aryl groups as described above. These groups may be further substituted as described above.

Therefore R can be hydrocarbyl, heterocarbyl, substituted hydrocarbyl substituted heterocarbyl and the like. The term hydrocarbyl as used herein means the monovalent moiety obtained upon removal of a hydrogen atom from a parent hydrocarbon. Representative of hydrocarbyl are alkyl of 1 to 20 carbon atoms, inclusive, such as methyl, ethyl, propyl, butyl, pentyl, hexyl, heptyl, octyl, nonyl, undecyl, decyl, dodecyl, octadecyl, nonodecyl, eicosyl, heneicosyl, docosyl, tricosyl, tetracosyl, pentacosyl and the isomeric forms thereof; aryl of 6 to 12 carbon atoms, inclusive, such as phenyl, tolyl, xylyl, naphthyl, biphenyl, tetraphenyl and the like; aralkyl of 7 to 12 carbon atoms, inclusive, such as benzyl, phenethyl, phenpropyl, phenbutyl, phenhexyl, napthoctyl and the like; cycloalkyl of 3 to 8 carbon atoms, inclusive, such as cyclopropyl, cyclobutyl, cyclopentyl, cyclobexyl, cycloheptyl, cyclooctyl and the like; alkenyl of 2 to 10 carbon atoms, inclusive, such as vinyl, allyl, butenyl, pentenyl, hexenyl, octenyl, nonenyl, decenyl, undececyl, dodecenyl, tridecenyl, pentadecenyl, octadecenyl, pentacosynyl and isomeric forms thereof. Preferably, hydrocarbyl has 1 to 20 carbon atoms, inclusive. The term substituted hydrocarbyl as used herein means the hydrocarbyl moiety as previously defined wherein one or more hydrogen atoms have been replaced with a chemical group which does not adversely affect the desired preparation of the product derivative. Representative of such groups are amino-, phosphino-, quaternary nitrogen (ammonium), quaternary phosphorous (phosphonium), hydroxyl, amide, alkoxy, mercapto, nitro, alkyl, halo, sulfone, sulfoxide, phosphate, phosphite, carboxylate, carbamate groups and the like. Carbodihydrazides are preferred, however other dihydrazides are within the scope of the invention, such as sulfonodihydrazides and phosphonic dihydrazides. Accordingly, adipic dihydrazide refers to H₂N—NH—C(═O)—(CH₂)₄—C(═O)—NH—NH₂.

Hydrophobic dihydrazides that are known to render HA more resistance to hyaluronidase degradation can be used in the formulation of the HA-matrix system so as to prolong the period of time over which nucleic acids are released. Likewise, Hydrophilic dihydrazides (Vercruysse K. P., Bioconj Chem., 8:686, 1997) can be used to shorten the period of time over which nucleic acids are released.

The term “moiety” refers to a part, portion or subunit of a larger compound.

The term “crosslinked” or “conjugated” refers to the attachment of two substances via any type of bond or force. A non-limiting list of specific means by which to crosslink two substances may include covalent bonds, ionic bonds or hydrogen bonds, van der Waals forces, ionic interactions and hydrophobic interactions.

### Embodiments of the Derivatized Hyaluronic Acid/DNA Microspheres

Without being bound by a single theory, it is believed that the microspheres of the invention include hyaluronic acid which is crosslinked to a dihydrazide wherein the dihydrazide portion of the molecule is further crosslinked to a nucleic acid; microspheres including such a molecule are within the scope of the invention. The microspheres of the invention include hyaluronic acid which has been derivatized with a dihydrazide. A nucleic acid is crosslinked to this derivative. The nucleic acid may be crosslinked to the dihydrazide derivatized hyaluronic acid molecule at any location on said dihydrazide derivatized hyaluronic acid molecule. Adipic dihydrazide is the preferred dihydrazide with which to derivatize hyaluronic acid, however, other dihydrazide molecules may be used for this purpose if the hyaluronic acid derivative which is produced may be crosslinked to a nucleic acid. In this application, microspheres including dihydrazide derivatized hyaluronic acid, crosslinked to a nucleic acid may be referred to as “microspheres of the invention”. The nucleic acids may be in the form of linear, single or double stranded DNA or RNA, however, in a preferred embodiment, the nucleic acid is plasmid DNA. Accordingly, a preferred embodiment of the invention includes microspheres comprising plasmid DNA conjugated to adipic dihydrazide derivatized hyaluronic acid. The microspheres of the invention may be further conjugated to other substances such as other small molecules, proteins and peptides. These additional substances may impart an additional therapeutic functionality upon the microspheres of the invention. The microspheres of the invention may be further conjugated to ligands which allow the microsphere to be targeted to a particular location in the patient. This location may be a particular cell type or organ. Furthermore, the additional conjugates may prevent or inhibit the microspheres of the invention from contacting certain cell types or organs.

The nucleic acids of the microspheres of the invention may encode a gene. This embodiment may include a gene for a growth factor; in preferred embodiments the growth factor is vascular epidermal growth factor (VEGF). A further, preferred embodiment may include said microspheres comprising plasmid DNA that encodes the VEGF gene. Microspheres that include said genes may also include, within the nucleic acid that contains the gene, additional nucleotides whose sequence causes expression of a protein or RNA, which corresponds to the gene, in a cell. The microspheres of the invention may include genes or nucleic acids which facilitate the practice of the invention, for example, the inclusion of an auxiliary gene whose expression causes the transferred nucleic acids to remain in the host cell for a longer period of time than in the absence of the auxiliary gene is within the scope of the invention. Auxiliary genes which increase or decrease the expression of the therapeutic gene, VEGF for example, may be included in the nucleic acids of the microspheres of the invention.

Accordingly, microspheres including adipic dihydrazide derivatized hyaluronic acid that is conjugated to plasmid DNA that encodes the VEGF gene is a preferred embodiment of the invention.

Yet another embodiment may include microspheres which include nucleic acids which have a nucleotide sequence of at least 70% identity to the reference nucleotide sequence set forth in SEQ ID NO. 1 as well as nucleic acids which have a nucleotide sequence which encodes a protein whose amino acid sequence has at least 70% identity to the reference amino acid sequence set forth in SEQ ID NO.2, wherein identity is determined using the BLASTN or BLASTP algorithms, respectively, where the parameters are selected to give the largest match between the respective sequences tested, over the entire length of the respective reference sequences.

In preferred embodiments, the microspheres of the invention have an average diameter of between about 15 μm to about 25 μm. However, microspheres of any size wherein the essential elements of the present invention are preserved are within the scope of this invention.

The preparation of nucleic acids which may be used in the present invention may be accomplished by any means which yields nucleic acids of sufficient quality and purity so as to allow the successful practice of the invention.

### Therapeutic Uses of Derivatized Hyaluronic Acid/DNA microspheres

The present invention include any embodiments wherein microspheres of the invention, may be administered to a subject, such as a human or animal, so as to cause a sustained transfer of the nucleic acid to cells of the subject. The use of the microspheres of the invention in the treatment of any medical condition wherein a sustained transfer of nucleic acids to the cells of a patient would provide a therapeutic effect are within the scope of the present invention. An induction of angiogenesis may be the therapeutic effect attained in these embodiments which may be used in the treatment of myocardial ischemia. In a preferred embodiment, microspheres including adipic dihydrazide derivatized hyaluronic acid, which is conjugated to plasmid DNA encoding the VEGF gene, are administered to a human patient for the purpose of inducing angiogenesis to treat myocardial ischemia.

The microspheres of the invention provide a high degree of versatility in terms of the types of medical conditions they may be used to treat. Simply substituting the type of nucleic acid to be delivered to a subject would be sufficient to adapt the microspheres of the invention to a newly discovered indication. Additional indications for which the microspheres of the invention may be employed, may include hemophilia. In these embodiments, a nucleic acid which comprises a gene whose product is involved in blood clotting is delivered to the cells of a patient. These genes may include Factor VIII and Factor IX.

### Formulations and administration

Microsphere formulations may be packaged in unit-dosage vials in freeze-dried powder form for subsequent shipment or storage. The powdered microspheres may then be reconstituted/resuspended in a diluent, such as sterile saline, and administered to a subject. Administration of the microspheres of the present invention may be accomplished by any means which delivers the microspheres to the location at which the therapeutic effect is needed. In preferred embodiments, the microspheres of the invention are delivered directly to the location at which they are needed to cause a therapeutic effect. In the treatment of myocardial ischemia, for example, the microspheres of the invention may be delivered to cardiac tissues by way of a catheter. The catheter may be inserted into the femoral artery, or any artery leading from the heart, and led up to the heart; once at the heart, the drug may be delivered. The microspheres may also be delivered to cardiac tissues by the insertion of a cardiac needle or shunt into the thoracic cavity of a subject. When the needle or shunt is proximal to the heart, the microspheres may be delivered.

### EXAMPLES

The invention may be better understood by reference to the following examples, which is provided by way of exemplification and not limitation.

### Example 1

### Formulation and Evaluation of Hyaluronic Acid (HA) DNA Delivery Microspheres

This example illustrates a method to synthesize and to evaluate HA DNA delivery microspheres. Specifically, the appearance of the microspheres as well as quality of the DNA recovered from a sustained release from the microspheres is analyzed. Further, this example illustrates the efficacy of the HA DNA delivery microspheres of this invention in the delivery of a β-galactosidase reporter gene to Chinese hamster ovary (CHO) cells in a cell culture.

### Materials and Methods

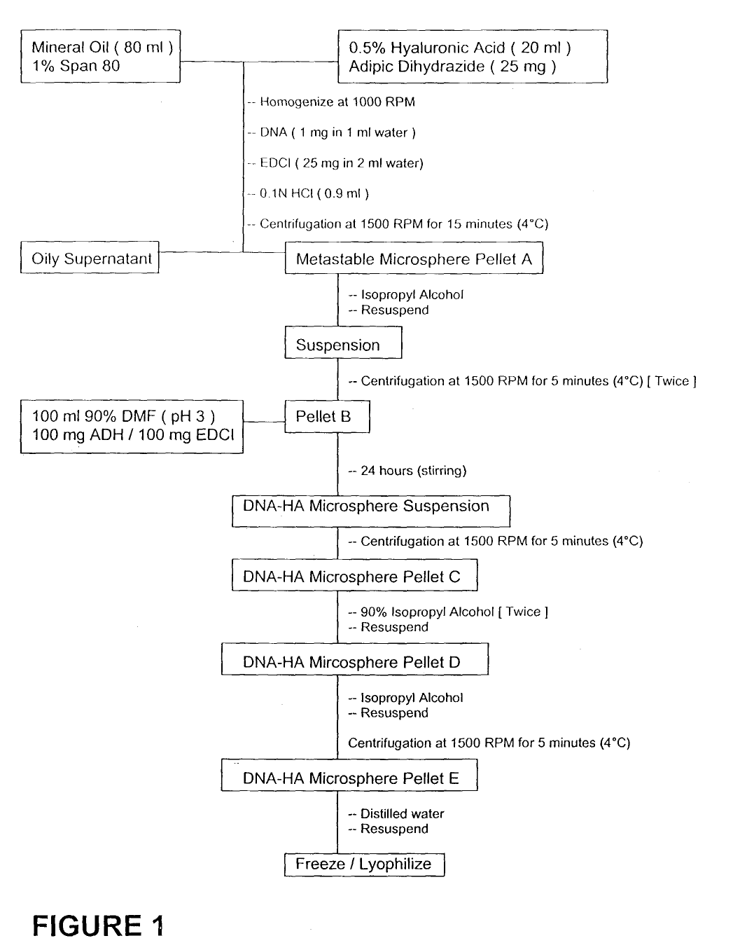

Formulation and visual evaluation of the Hyaluronic Acid DNA delivery microspheres. The preparation of DNA microspheres is outlined schematically in FIG. 1. Briefly, 25 mg of adipic dihydrazide (ADH) was dissolved in 25 ml of 0.5%(w/v) hyaluronic acid (HA) solution. This solution was homogenized with 80 ml of mineral oil (with 1 ml of Span 80 dissolved) using a mixer with the impeller rotating at 900 to 1000 RPM. Span 80 is a nonionic surfactant, sorbitan monooleate. Upon the formation of a milky emulsion, 1 ml of DNA solution (1 mg/ml) was slowly delivered into the emulsion while mixing. This was followed by the addition of 2 ml of ethyl-3[3-dimethyl amino] propyl carbodimide (EDCL) and 0.9 ml of 0.1 N HCl. The emulsion was then centrifuged at 1500 RPM for 15 minutes at 4° C. The sediment (Pellet A) was recovered and resuspended in isopropyl alcohol; and it was then centrifuged at 1500 RPM for 5 minutes at 4° C. This procedure was repeated once. The sediment (Pellet B) was collected and resuspended in 100 ml of a solvent mixture of dimethyl formamide (DMF) and water (90:10) for 4 to 24 hours. Upon completion of the crosslinking, the DNA-HA microspheres were centrifuged at 1500 RPM for 5 minutes at 4° C. This procedure was repeated once. The sediment (Pellet D) was collected and resuspended in isopropyl alcohol. After a final centrifugation of 1500 RPM for 5 minutes at 4° C., the sediment (Sediment F) was resuspended in distilled water. This DNA-HA microsphere suspension was then frozen and lyophilized.

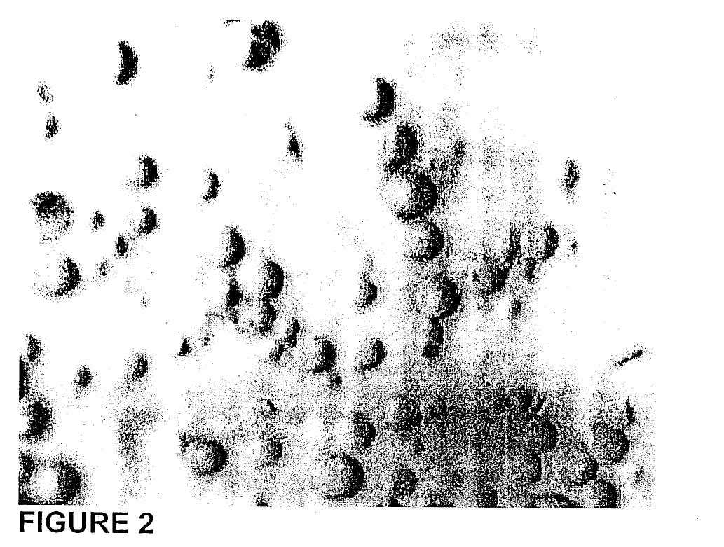

As illustrated in FIG. 2, the appearance of the microspheres were evaluated using a light microscope. The sizes of most microspheres are between 15 μm and 25 μm in diameter.

The sequence of the commercially available plasmid from Invitrogen, pCDNA 3.1/GS into which the VEGF gene was inserted is set forth in SEQ ID NO.3. This plasmid causes VEGF expression to be driven by the CMV promoter.

Controlled DNA-release study of DNA-HA microspheres in hyaluronidase. Microspheres prepared by the method described in the previous section were tested for controlled release kinetics. This experiment demonstrates that the microspheres of the invention gradually release the crosslinked nucleic acids; in therapeutic applications this gradual release allows the sustained transfer of the nucleic acids to a subjects cells. A sample of the microspheres was incubated in a container with 1 ml of phosphate buffer and saline(PBS) containing hyaluronidase, at a concentration of 10 units/ml. At various time intervals, the PBS/hyaluronidase mixture was evacuated from the container and replenished with a fresh aliquot of PBS/hyaluronidase buffer. The mixtures which were evacuated from the container were tested for the presence of DNA. The DNA obtained during this controlled release study was tested for its ability to transfect chinese hamster ovary(CHO) cells in culture. Table 1 is a report of the number of transfected cells at various times after exposure to the DNA.

TABLE 1
Longevity of DNA Release from HA-Microspheres and
Magnitude of CHO Cell Transfection

| Time (days) | Relative Level of Transfection |
|---|---|
| 2 | +++++ |
| 5 | +++++/++++ |
| 9 | +++++/++++ |
| 12 | +++/++ |
| 16 | ++ |
| 25 | +++/++ |
| 30 | ++ |

+++++ = 400-500
++++ = 300-400
+++ = 200-300
++ = 100-200
+ = <100

+++++=400-500

++++=300-400

+++=200-300

++=100-200

+=<100

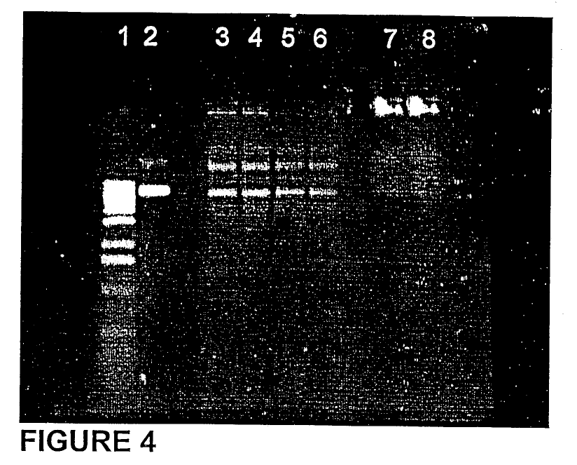

Evaluation of the quality of DNA in DNA-HA microspheres before and after controlled DNA-release. The DNA samples collected during the course of the controlled release study were loaded into an agarose gel (FIG. 4, middle 4 lanes). A small sample of the microspheres was incubated in Tris-EDTA buffer for hydration. The microsphere suspension was also loaded into the agarose gel (FIG. 4, right 2 lanes). The DNA released from the microspheres remained intact; whereas the whole microspheres which were loaded failed to migrate into the gel. This experiment demonstrates that nucleic acids released from the microspheres of the invention are intact and not fragmented or degraded.

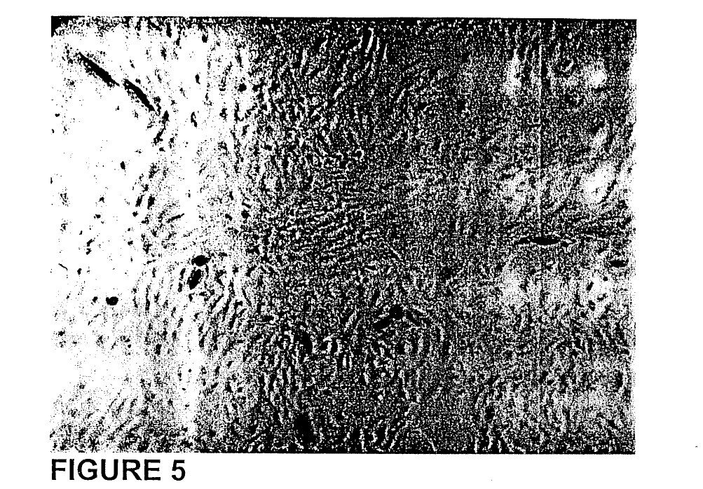

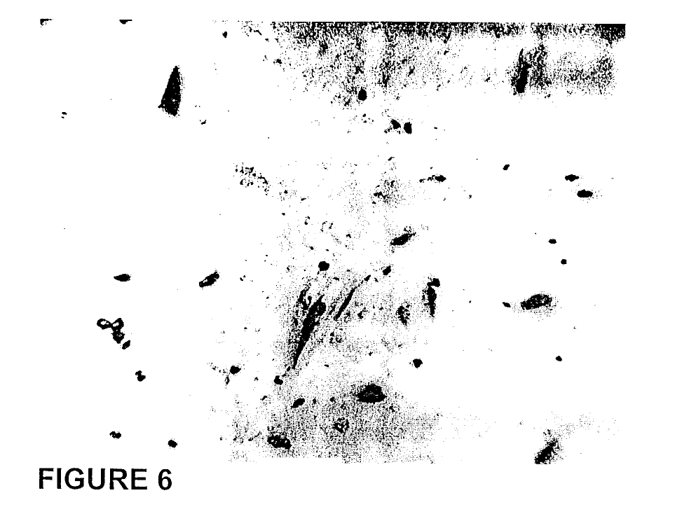

Transfection of CHO cells with a β-galactosidase reporter gene in DNA-HA microspheres or recovered from controlled release experiments. A microsphere suspension, with a Plasmid containing a β-galactosidase reporter gene incorporated was deposited in a chinese hamster ovary (CHO) cell culture. FIG. 5 is a photograph of cells transfected for 72 hours with microspheres which were crosslinked for 24 hours. FIG. 6 is a photograph of cells transfected for 72 hours with microspheres which were crosslinked for 4 hours. This experiment is a demonstration of the ability of the microspheres of the invention to deliver nucleic acids to a whole cell.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

It is further to be understood that all base sizes or amino acid sizes, all synthetic antibody concentrations and all molecular weight or molecular mass values, are approximate, and are provided for description.

All patents, applications, publications, test methods, literature, and other materials cited herein are hereby incorporated by reference.

What is claimed:

1. A microsphere comprising dihydrazide derivatized hyaluronic acid crosslinked to a nucleic acid.

2. The microsphere of claim 1 wherein the dihydrazide is adipic dihydrazide.

3. The microsphere of claim 1 wherein said nucleic acid is selected from the group consisting of plasmid DNA, linear single stranded DNA, linear double stranded DNA, RNA, and an oligonucleotide.

4. The microsphere of claim 1 wherein the nucleic acid is plasmid DNA and the plasmid DNA encodes a protein which, when present in the body of a subject, causes angiogenesis or causes the production of a substance which causes angiogenesis.

5. A microsphere comprising dihydrazide derivatized hyaluronic acid crosslinked to a nucleic acid wherein said nucleic acid comprises the nucleotide sequence set forth in SEQ ID NO: 1.

6. A microsphere comprising dihydrazide derivatized hyaluronic acid crosslinked to a nucleic acid wherein said nucleic acid has a nucleotide sequence of at least 95% identity to SEQ ID NO. 1, wherein identity is determined using the BLASTN algorithm, where the parameters are selected to give the largest match between the sequences tested over the entire length of SEQ ID NO. 1.

7. The microsphere of claim 6 wherein said nucleic acid is selected from the group consisting of plasmid DNA, linear single stranded DNA, linear double stranded DNA, RNA, and an oligonucleotide.

8. The microsphere of claim 6 wherein the nucleic encodes a protein which, when present in the body of a subject, causes angiogenesis or causes the production of a substance which causes angiogenesis.

9. A method of introducing a nucleic acid into a cell of a subject comprising transfecting the cell with a nucleic acid from the microsphere of claim 6.

10. A method of treating a subject in need of increased cardiac angiogenesis comprising contacting the heart of the subject with the microsphere of claim 8, wherein said contacting results in increased angiogenesis.

11. The method of claim 12 wherein the subject has myocardial ischemia, wherein said contacting results in treatment of the myocardial ischemia.

12. A cell which is transfected by a method which comprises introducing into the cell a nucleic acid from a microsphere of claim 1.

13. A cell which is transfected by a method which comprises introducing into the cell a nucleic acid from a microsphere of claim 6.

14. A microsphere comprising dihydrazide derivatized hyaluronic acid crosslinked to a nucleic acid, wherein said nucleic acid, has a nucleotide sequence which encodes a protein of at least 95% identity to SEQ ID NO.2, wherein identity is determined using the BLASTP algorithm, where the parameters are selected to give the largest match between the sequences tested over the entire length of SEQ ID NO.2.

15. A microsphere comprising dihydrazide derivatized hyaluronic acid crosslinked to a nucleic acid wherein said nucleic acid encodes a protein which comprises the amino acid sequence set forth in SEQ ID NO: 2

16. A method of treating myocardial ischemia in a subject comprising contacting the heart of the subject with a microsphere comprising dihydrazide derivatized hyaluronic acid crosslinked to a plasmid whose nucleotide sequence comprises that set forth in SEQ ID NO. 1, wherein said contacting results in treatment of the myocardial ischemia.


SEQUENCE LISTING (3 sequences)

SEQ ID NO: 1 - DNA, 573 bp, human
atgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcgg

SEQ ID NO: 2 - protein, 191 aa, human
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 3 - DNA, 4597 bp, Artificial Sequence
  pCDNA3.1/GS vector by Invitrogen Corporation
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagctcgcccttcaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggaagggcgagcttcgaggtcacccattcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagcggatacatatttgaatgtatttagaaaaataaacaaataggggttccgcgcacatttccccgaaaagtgccacctgacgtc